(19) United States
(12) Patent Application Publication
(10) Pub. No.: US 2019/0125934 A1
(43) Pub. Date: May 2, 2019

(54) TOUGH HYDROGEL COATING AND METHOD OF MANUFACTURE

(71) Applicant: Massachusetts Institute of Technology, Cambridge, MA (US)

(72) Inventors: Xuanhe Zhao, Allston, MA (US); German Alberto Parada Hernandez, Cambridge, MA (US); Hyunwoo Yuk, Cambridge, MA (US)

(21) Appl. No.: 15/794,432

(22) Filed: Oct. 26, 2017

(51) Int. Cl. A61L 29/14; A61L 29/08; A61L 31/10; A61L 31/14; A61L 31/16; A61L 29/16; G01N 21/80

(52) U.S. Cl. CPC A61L 29/145; A61L 29/085; A61L 31/10; A61L 31/145; A61L 31/16; A61L 29/16; G01N 2021/7756; A61L 2300/606; A61L 2420/08; A61L 2420/02; A61L 2420/06; A61L 2300/61; A61L 2400/10; G01N 21/80

(57) ABSTRACT

Hydrogel-substrate laminate structures and tough biocompatible hydrogel coatings for various equipment such as medical devices and underwater equipment, in which robust interfaces are formed between the hydrogel coatings and the substrate/equipment surface(s). The hydrogel coatings provide a highly-hydrated, ultra-low friction structure that does not rupture or delaminate under stress. The hydrogel coatings may further incorporate a variety of therapeutic agents and/or sensing mechanisms to provide for environmental sensing and therapeutic agent release.

This invention was made with Government support under Grant No. N00014-14-1-0619 awarded by the Office of Naval Research, and Contract No. W911NF-13-D-0001 awarded by the Army Research Office. The Government has certain rights in the invention.

### FIELD OF THE INVENTION

The present invention relates to a tough hydrogel coating and method of manufacture wherein robust bonds are formed between the hydrogel and the coated surface. One or more therapeutic agents and/or sensing materials may further be incorporated into the hydrogel coating to provide a coated structure having therapeutic agent release and/or environmental sensing capabilities.

### BACKGROUND OF THE INVENTION

Hydrogels are hydrophilic polymeric materials capable of holding large amounts of water in their three-dimensional networks. They are typically made using natural polymers (e.g., collagen and alginate) or synthetic polymers (e.g., poly(vinyl alcohol) (PVA) and poly(acrylic acid) (PAA)). Depending on the nature of the hydrogel network, they can be categorized as either “physical” hydrogels, which means that the network formation is reversible, or “chemical” hydrogels, which means that the network formation is irreversible and is formed by covalent cross-links. Due to their high water content, porosity, and soft consistency, hydrogels closely resemble natural living tissue. These properties, along with their generally good biocompatibility and ease of fabrication, make hydrogels desirable for use in a number of biomedical applications. In particular, hydrogels have been widely used in biomedical applications such as drug delivery, tissue engineering, tissue bulking agents, and contact lenses. The use of hydrogels has more recently been explored as material candidates for medical tubing and catheters, control elements in fluidic devices, antifouling coatings, and soft electronics and machines. However, despite the many beneficial properties that hydrogels possess, their potential for use in these applications has been significantly hampered by their low mechanical robustness, permeability to various molecules, and weak hydrogel-solid interfaces.

For example, hydrogels possess low tensile strength which limits their use in applications requiring load-bearing. In such load-bearing applications, hydrogels are typically unable to maintain their shape and function in the long-term. Further, the strength and fracture toughness of common hydrogels are usually much lower than the corresponding elastomers (e.g., silicone rubbers and latex) traditionally used for the aforementioned applications. In addition, most hydrogels are brittle and possess very low stretchability, with typical fracture energies of hydrogels being about 10 J m ⁻² as compared with ˜1,000 J m ² for cartilage and ˜10,000 J M⁻² for natural rubbers. Moreover, formation of weak hydrogel-solid interfaces results in a failure to integrate soft hydrogels and rigid components with adequate functionality and reliability. For example, coatings using conventional hydrogels can easily fracture and delaminate upon application of stress. In drug delivery applications, it may be problematic to load and effectively deliver certain drugs from hydrogels. In particular, in the case of hydrophobic drugs, the high water content and high porosity of most hydrogels can result in rapid drug release rather than a desired slower and sustained release of the drug. Further, hydrogels suffer from high permeability to small molecules, which presents a further challenge in the field.

Various attempts have been made to address these limitations. However, in view of the great potential that hydrogels possess, further improvements are still needed.

### SUMMARY OF THE INVENTION

Embodiments of the present invention combine the permeable, compliant, tunable, and slippery nature of tough hydrogels with the non-permeable and relatively rigid properties of commonly-used engineering materials (e.g., elastomers, plastics, glass, ceramics and metals). Hydrogel-substrate combined structures are provided through robust bonding between the hydrogels and a substrate (e.g., substrates fabricated of elastomers, plastics, glass, ceramics, metals, etc.) resulting in a highly-hydrated, ultra-low friction laminate structure that is also tough enough to handle manipulation without rupture or delamination of the coating material. Of particular interest are structures where one or more thin hydrogel layers are coated onto elastomer substrates, termed hydrogel-elastomer laminates. According to methods of the present invention, the thickness of the hydrogel coating layer(s) in these laminates can be tuned to match a wide range of mechanical properties from pure elastomer (corresponding to a thinner hydrogel layer) to pure hydrogel (corresponding to a thicker hydrogel layer), while maintaining a surface with a very low coefficient of friction. In addition, the hydrogel-elastomer laminates are impermeable to small molecules across the laminate structure, enable controlled release of a variety of therapeutic agents, and provide for sensing of various stimuli surrounding the laminate structure. The present invention further provides hydrogel coated medical devices which can be used for environmental sensing and therapeutic agent release while reducing the surface friction of these devices.

According to one aspect, the present invention provides a hydrogel-substrate laminate comprising a layer of substrate having a top surface and a bottom surface, the substrate selected from elastomers, plastics, glass, ceramics and metals; at least one hydrogel coating layer disposed on one or more of the top surface and the bottom surface of the substrate; and one or more therapeutic agent disposed within the at least one hydrogel coating layer, and/or one or more sensing material disposed within the at least one hydrogel coating layer.

Embodiments according to this aspect can include one or more of the following features. A first hydrogel coating layer can be disposed on the top surface of the substrate layer, and a second hydrogel coating layer can be disposed on the bottom surface of the substrate layer. The layer of substrate can be impermeable to small molecules, a first therapeutic agent can be disposed within the first hydrogel coating layer, and a second therapeutic agent different than or the same as the first therapeutic agent can be disposed within the second hydrogel coating layer. The layer of substrate can be impermeable to small molecules, a first sensing material can be disposed within the first hydrogel coating layer, and a second sensing material different than or the same as the first sensing material can be disposed within the second hydrogel coating layer. The layer of substrate can be impermeable to small molecules, at least one therapeutic agent can be disposed within the first hydrogel coating layer, and at least one sensing material can be disposed within the second hydrogel coating layer. At least on therapeutic agent and at least one sensing material can be disposed within the at least one hydrogel coating layer, the at least one therapeutic agent and the at least one sensing material can be in communication, and release of the at least one therapeutic agent from the at least one hydrogel coating layer is controlled by one or more environmental conditions sensed by the at least one sensing material. A controller or the like can be provided so as to regulate release of the at least one therapeutic agent from the at least one hydrogel coating layer based upon the one or more sensed environmental conditions. The at least one therapeutic agent can be disposed within a first hydrogel coating layer, and the at least one sensing material can be disposed within a second hydrogel coating layer, wherein the first and second hydrogel coating layers are separated from each other by the layer of substrate. The thickness of the hydrogel coating layer can be predetermined to provide a hydrogel-substrate laminate having mechanical properties ranging from mechanical properties of the pure substrate to mechanical properties of the pure hydrogel. The at least one hydrogel coating layer can comprise a physically crosslinked dissipative polymer network selected from PVA, collagen, gelatin, agar, agarose, dextran, alginate, hyaluronan and chitosan; and a covalently crosslinked stretchy polymer network selected from polyacrylamide (PAAm), polyethylene glycol (PEG), polyethylene glycol derivatives, polyvinyl alcohol, poly-N,N-dimethylacrylamide (DMMA), polyacrylamide derivatives, and polyzwitterionic monomers. The substrate can be an elastomer material, and the stretchy polymer network in the hydrogel coating layer can be covalently grafted to elastomer chains of the elastomer material.

According to another aspect, the present invention provides a hydrogel coated medical device comprising a medical device having an outer surface; at least one hydrogel coating layer disposed on at least a portion of the outer surface of the medical device; and one or more therapeutic agent disposed within the at least one hydrogel coating layer to provide therapeutic agent release into an environment surrounding the at least one hydrogel coating layer, and/or one or more sensing material disposed within the at least one hydrogel coating layer to provide sensing of one or more environmental conditions surrounding the at least one hydrogel coating layer.

Embodiments according to this aspect can include one or more of the following features. The hydrogel coated medical device has a coefficient of friction less than the medical device without the hydrogel coating layer. At least on therapeutic agent and at least one sensing material can be disposed within the at least one hydrogel coating layer, wherein the at least one therapeutic agent and the at least one sensing material are in communication, and wherein release of the at least one therapeutic agent from the at least one hydrogel coating layer is controlled by one or more environmental conditions sensed by the at least one sensing material. A controller or the like can be provided so as to regulate release of the at least one therapeutic agent from the at least one hydrogel coating layer based upon the one or more sensed environmental conditions. A plurality of different therapeutic agents can be disposed within the at least one hydrogel coating layer so as to be independently released from the at least one hydrogel coating layer.

According to another aspect, the present invention provides a method for fabricating a hydrogel-substrate laminate comprising activating at least one surface of a pristine substrate material, the substrate material selected from elastomers, plastics, glass, ceramics and metals; coating at least one surface of the activated substrate material with a layer of hydrogel precursor solution; curing the hydrogel precursor solution to thereby form a tough, double network hydrogel coating layer of controllable thickness firmly grafted on the at least one surface of the activated substrate material.

Embodiments according to this aspect can include one or more of the following features. The hydrogel precursor solution can contain one or more therapeutic agent and/or one or more sensing material. Prior to curing the hydrogel precursor solution, desired mechanical properties of the hydrogel-substrate laminate ranging from mechanical properties of the pure substrate to mechanical properties of the pure hydrogel can be determined, based on the desired mechanical properties, a corresponding thickness of the hydrogel coating layer can be determined, and at least one spacer can be placed on the layer of hydrogel precursor solution to control a thickness of the hydrogel coating layer. Activating at least one surface of a pristine substrate material can comprise surface functionalization of the substrate surface. The hydrogel coating layer can be formed by grafting hydrogel polymer chains onto the substrate surface by generation of free radicals at the substrate-hydrogel interface.

Other systems, methods and features of the present invention will be or become apparent to one having ordinary skill in the art upon examining the following drawings and detailed description. It is intended that all such additional systems, methods, and features be included in this description, be within the scope of the present invention and protected by the accompanying claims.

### BRIEF DESCRIPTION OF THE DRAWINGS

The accompanying drawings are included to provide a further understanding of the invention, and are incorporated in and constitute a part of this specification. The components in the drawings are not necessarily to scale, emphasis instead being placed upon clearly illustrating the principles of the present invention. The drawings illustrate embodiments of the invention and, together with the description, serve to explain the principals of the invention.

FIG. 1A schematically illustrates the structure of an impermeable hydrogel laminate with a detailed hydrogel-elastomer interface network structure according to an embodiment of the present invention, FIG. 1B schematically illustrates sensing capabilities for different environments of an impermeable hydrogel laminate according to an embodiment of the present invention, FIG. 1C schematically depicts release capabilities to different environments of an impermeable hydrogel laminate according to an embodiment of the present invention, and FIG. 1D is an image of an impermeable hydrogel laminate according to an embodiment of the present invention in comparison to a hydrogel sheet.

FIG. 2 schematically illustrates a hydrogel coating process by dip-coating a device into a hydrogel precursor solution, according to an embodiment of the present invention.

FIGS. 3A-K illustrate exemplary coated medical devices, with FIGS. 3A-C illustrating a hydrogel coated silicone medical tubing according to an embodiment of the present invention which is manipulated and bent to test hydrogel toughness and bonding, FIGS. 3D-F illustrating a hydrogel coated latex condom according to an embodiment of the present invention which is inflated to test hydrogel toughness and bonding, FIGS. 3G-H illustrating hydrogel coated Foley Catheter according to an embodiment of the present invention which is inflated to test hydrogel toughness and bonding, and FIGS. 3I-K illustrating cross-sectional microscopic images of each of the coated devices of FIGS. 3A, 3D and 3G, respectively.

FIG. 4 illustrates bacterial adhesion experimental results, particularly comparison of controls (latex and glass) to hydrogel laminates after a 24 hour incubation period.

FIGS. 5A-D illustrate tensile properties of hydrogel and impermeable hydrogel laminates, with FIG. 5A illustrating nominal stress versus stretch curves of elastomer, hydrogel, and laminates of varying elastomer-to-hydrogel thickness, FIG. 5B illustrating an embodiment of the present invention hydrogel-elastomer interface which does not delaminate upon stretching up to seven times, FIG. 5C showing a microscope image of a nonpermeable hydrogel laminate with H_{E}/H_{G}=2.0 according to an embodiment of the present invention, and FIG. 5D showing an image of a nonpermeable hydrogel laminate with H_{E}/H_{G}=0.08 according to an embodiment of the present invention.

FIGS. 6A-E illustrate diffusion characteristics of impermeable hydrogel-elastomer laminate, with FIG. 6A schematically illustrating a two-chamber experimental setup, FIG. 6B showing diffusion curves of rhodamine B dye across an elastomer, a hydrogel slab, and impermeable hydrogel laminate samples according to embodiments of the present invention, FIG. 6C showing an image of an impermeable hydrogel laminate according to an embodiment of the present invention before a diffusion experiment. FIG. 6D showing an image of an impermeable hydrogel laminate according to an embodiment of the present invention after a diffusion experiment, wherein the dye does not diffuse past the elastomer layer, and FIG. 6E showing an image of a pure hydrogel sheet after the diffusion experiment, wherein the dye diffuses across the sample.

FIGS. 7A-D illustrate release characteristics of hydrogel laminates to different environments according to an embodiment of the present invention, with FIG. 7A schematically depicting a two-chamber experimental setup to study drug release from impermeable hydrogel laminates to different environments, FIG. 7B showing release profiles of rhodamine B and green food dye to the separate chambers with Fickian diffusion power law fits, FIG. 7C showing a loaded impermeable hydrogel laminate according to an embodiment of the present invention before a release experiment, with different drugs loaded into hydrogel layers on either side of an elastomer layer, and FIG. 7D showing the impermeable hydrogel laminate of FIG. 7C after the release experiment.

FIGS. 8A-D illustrate sensing capabilities of hydrogel laminates according to an embodiment of the present invention, where FIG. 7A depicts a color response chart for a pH sensitive dye used for sensing on both sides of the impermeable laminates, and FIGS. 8B-E illustrate the sensing of different pH conditions on different sides of the impermeable hydrogel laminate according to an embodiment of the present invention, wherein the hydrogel coatings show different colorimetric responses based on the pH of the solution that the hydrogel layer was exposed to.

### DETAILED DESCRIPTION

The following definitions are useful for interpreting terms applied to features of the embodiments disclosed herein, and are meant only to define elements within the disclosure.

As used herein, the term “hydrogel laminate” refers to a structure composed of one or more thin hydrogel coating on a substrate (e.g. elastomer layer, plastic layer, glass layer, ceramic layer, metal layer, etc.). The hydrogel coating can be on both sides of the substrate or on one side.

As used herein, the term “pristine”, such as when referring to a pristine elastomer or pristine surface, means such a surface prior to any surface treatment or modification.

The present invention generally provides hydrogel coatings and methods of fabrication. The hydrogel coatings are robust, highly stretchable (provided the substrate is stretchable), and impart ultralow surface coefficients of friction on the surface that is coated. In addition, the composition and thickness of the hydrogel coating can be controlled to tune the stiffness of the hydrogel without sacrificing stretchability. In particular, the present invention provides a hydrogel coating layer that is firmly grafted to a surface and which consists of a crosslinked double network, which is in contrast with conventional methods which are limited to single materials and grafting polymer chains. As such, the present invention hydrogel coatings can be made of controllable thickness, and can be tuned to provide a number of predetermined desired characteristics. If desired, therapeutic agents and/or sensing mechanisms/substances may be incorporated into the hydrogel coatings to provide for release of the therapeutic agents and/or sensing of environmental conditions in which the hydrogel coating is disposed.

According to one embodiment, the hydrogel coating is bonded to a material to provide a laminate structure. In an exemplary embodiment, an impermeable material, such as an elastomer layer, is sandwiched between two opposing hydrogel layers. The resulting hydrogel elastomer laminate retains the high water content and slippery surface properties of the hydrogel and, unlike the hydrogel material itself, does not allow diffusion of various types of compounds (e.g., small-molecule chemicals, biomolecules, and nanoparticles) across the laminate structure due to the presence of the impermeable material layer. If desired, one or more therapeutic agents can be disposed within one or more of the hydrogel layers to provide therapeutic agent release, and/or one or more sensing mechanisms/substances can be disposed within the one or more hydrogel layers to provide sensing of various environmental conditions (e.g., pH, temperature, etc.) on one or more sides of the laminate structure.

The hydrogel is formed of a physically crosslinked dissipative polymer network and a covalently crosslinked stretchy polymer network. The covalently crosslinked polymer network is characterized in its high deformability, while the dissipative polymer network is characterized in its ability to dissipate significant mechanical energy under deformation. These two components (i.e., stretchy polymer networks and dissipative polymer network) are interpenetrated with each other after curing in such a way that they work synergistically, with the stretchy polymer networks functioning to maintain the integrity of the material, and the dissipative polymer network (e.g. ionic crosslinks, fiber filler, etc.) providing mechanical energy dissipation when the whole polymer network undergoes deformation.

The stretchy polymer networks can be selected from any known stretchy (preferably long-chain) polymer networks. Since the present hydrogels can be used in a wide variety of biomedical applications, the polymers used in the present invention are preferably biocompatible. Of course, it is to be understood that for non-biomedical applications, it would not be necessary to utilize only biocompatible polymer materials. In general, molecular weight determines the stretchability of the network, with larger molecular weights typically resulting in higher stretchability. As such, when high stretch is desired, polymers having a high molecular weight associated with higher stretchability are typically used to provide the stretchy polymer networks. Some examples of stretchy polymer networks include, but are not limited to, polyacrylamide (PAAm), polyethylene glycol (PEG) and polyethylene glycol derivatives (acrylated PEG, methacrylated PEG, PEG norbornene, PEG diacrylate, PEG dimethacrylate), polyvinyl alcohol, poly-N,N-dimethylacrylamide (DMMA), and polyzwitterionic monomers (sulfobetaine methacrylate, carboxybetaine methacrylate, phosphorylcholine methacrylate, and other methacrylate derivatives). According to an exemplary embodiment, these long-chain polymer networks are covalently crosslinked.

The dissipative component can, likewise, be any such known components. For example, according to various embodiments, mechanical dissipation is incorporated in the present materials by including ionic crosslinks, pull-out fibers, and/or transformation domain(s) in the polymer chains. In some embodiments, the dissipative component comprises dissipative polymer networks. According to particularly preferable embodiments, the dissipative material is reversibly dissipative, which means that the material can reform after damage to at least partially heal the strength of the material. Some examples of the dissipative components include, but are not limited to, PVA, collagen, gelatin, agar, agarose, dextran, alginate, hyaluronan and chitosan, which are all biocompatible materials. Such materials preferably contain reversible crosslinks, which enables the kinetics of zipping and unzipping of the dissipative materials (where the dissipative network acts to “unzip”—i.e., break—and “zip”—i.e., heal—the network), inhibits the propagation of cracks, and enhances anti-fatigue performance of the material. Thus, the reversible crosslinks not only provide energy dissipation from the breakage of the crosslinks, but also ensure the anti-fatigue performance due to reforming (i.e., healing) of the damaged crosslinks. Exemplary dissipative components can include alginate reversibly crosslinked by calcium sulfate, hyaluronan reversibly crosslinked by iron (III) chloride, and chitosan reversibly crosslinked by sodium tripolyphosphate. Of course, other combinations are possible and could be determined by one skilled in the art.

According to embodiments of the present invention, the stretchy polymer network in the hydrogel is covalently grafted to the elastomer chains of the elastomer material (or to the components of the other substrate materials, if utilized) to achieve robust bonding between the hydrogel and the elastomer layer. These robust bonds are formed by chemically anchoring the stretchy polymer in the tough hydrogels onto the surfaces of the elastomer layer. This chemical anchoring enables stable bonding of hydrogels having relatively high intrinsic adhesion energy and large enhanced surface toughness from the dissipative networks contained therein. Preferably, robust bonding of the hydrogels onto the elastomer layer (or other substrate material) is achieved by surface functionalization (i.e., modifying the surface using physical, chemical or biological mechanisms) of the elastomer layer surface(s). In preferred embodiments, the hydrogel polymer chains are grafted onto the solid surface by generation of free radicals at the solid-hydrogel interface.

Exemplary materials that can be used for the elastomer layer include silicone rubbers, such as polydimethyl siloxane (PDMS, Dow Corning®) and Ecoflex® (Smooth-On), latex, polyurethanes, and other natural or synthetic rubbers.

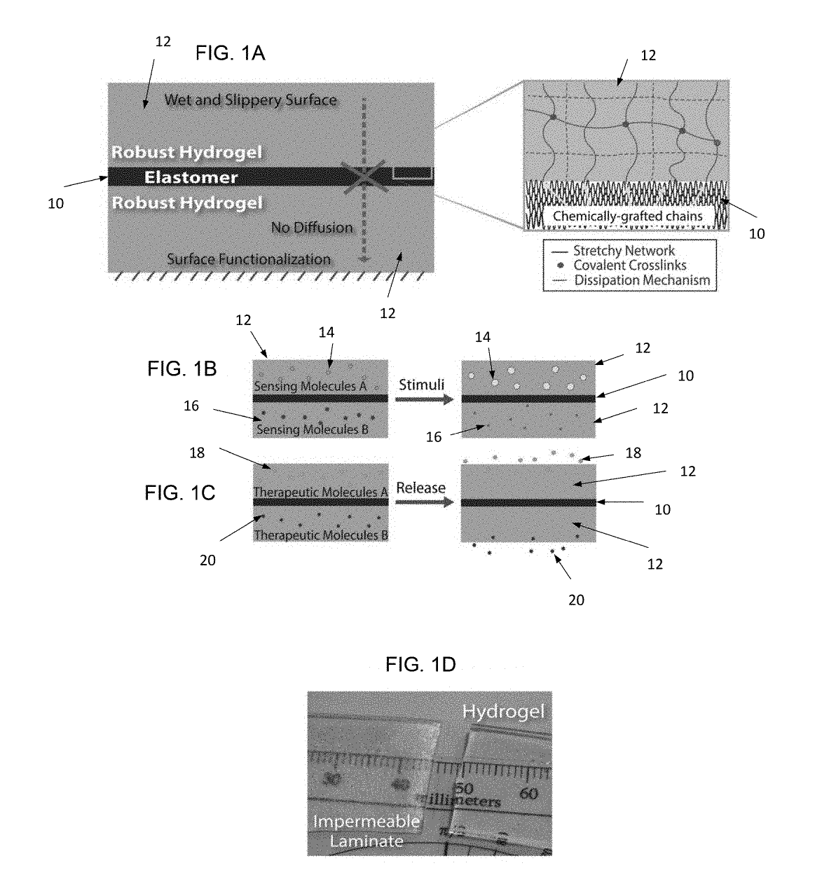

FIG. 1A illustrates an exemplary design strategy for an impermeable robust hydrogel laminate according to an embodiment of the invention. As shown, a layer of an impermeable elastomer 10 is sandwiched between two layers of a stretchable and robust hydrogel 12. Prior to coating the elastomer layer 10 with the hydrogel 12, the surface(s) of the elastomer layer 10 are activated. Any surface activating material, such as benzophenone or other radical sources (redox initiators such as cumene hydroperoxide/tetraethylenepentamine, tent-butyl hydroperoxide/tetraethylenepentamine, potassium persulfate/potassium metabisulfite and similar combinations), may be used to activate the surfaces of the elastomer layer 10. For glass, metals and ceramics, 3-(trimethoxysilyl)propyl methacrylate or other acrylated silane, methacrylated silane or heterobifunctional molecule with a acrylate or methacrylate moiety with a may be used to activate the surfaces. Such activation enables covalent grafting of the stretchy networks of the hydrogels onto the elastomer chains, for example, as illustrated in the right inset of FIG. 1A.

According to one embodiment, one or more sensing or stimuli-responsive mechanisms/molecules may be incorporated into the hydrogel 12 to monitor environmental conditions (e.g., pH, temperature, and biomolecule concentration) surrounding the hydrogel laminate (particularly adjacent the hydrogel layer in which the stimuli-responsive mechanisms/molecules are disposed). For example, as depicted in FIG. 1B, a plurality of different sensing molecules (sensing molecules A 14 and sensing molecules B 16) may be incorporated into each of the opposing hydrogel layers to monitor environmental conditions on opposing sides of the hydrogel laminate. For example, sensing molecules A 14 may sense the pH on one side of the laminate, while sensing molecule B 16 may monitor the temperature on the other side of the laminate. It is to be understood that the present invention is not limited as such. For example, if desired, sensing or stimuli-responsive molecules may be incorporated into only one of the hydrogel layers, or identical sensing or stimuli-responsive molecules can be incorporated into each of the hydrogel layers. Further, a plurality of different sensing or stimuli-responsive molecules may be incorporated into a single hydrogel layer if they are capable of maintaining their intended function when combined as such (i.e., they do not negatively interact with each other in a way that alters their intended sensing capabilities).

According to another embodiment, a variety of different types of therapeutic agents and functional molecules may be incorporated into the hydrogel 12 to provide release of the therapeutic agents and functional molecules into the environment surrounding the hydrogel laminate. For example, as shown in FIG. 1C, different types of therapeutic agents and functional molecules (therapeutic molecules A 18 and therapeutic molecules B 20) are incorporated into each of the opposing hydrogel layers to provide for release of different therapeutic molecules on opposing sides of the hydrogel laminate. Such therapeutic molecules may be independently released from each hydrogel layer. It is to be understood that the present invention is not limited as such. For example, if desired, therapeutic agents and functional molecules can be incorporated into only one of the hydrogel layers, or identical therapeutic agents and functional molecules can be incorporated into each of the hydrogel layers. Further, a plurality of different therapeutic agents and functional molecules may be incorporated into a single hydrogel layer if they are capable of maintaining their intended therapeutic effects/functions when combined as such.

According to another embodiment, a combination of sensing or stimuli-responsive molecules/mechanisms and therapeutic agents/functional molecules are incorporated into the hydrogel laminate. For example, one of the hydrogel layers may incorporate sensing or stimuli-responsive molecules/mechanisms while another hydrogel layer may incorporate therapeutic agents/functional molecules. As such, environmental conditions may be monitored on one side of the hydrogel laminate, while therapeutic agents/functional molecules may be released on another side of the hydrogel laminate. In some embodiments, the sensing or stimuli-responsive molecules/mechanisms and the therapeutic agents/functional molecules may be combined into the same hydrogel layer if the components maintain their intended sensing/stimuli-responsive and therapeutic effects/functions when combined as such. The hydrogel laminate can further be configured such that the sensing or stimuli-responsive molecules/mechanisms are in communication with the therapeutic agents/functional molecules to provide for release of the therapeutic agents/functional molecules if certain environmental conditions are detected by the sensing or stimuli-responsive molecules/mechanisms.

As shown in FIG. 1D, a hydrogel-elastomer-hydrogel laminate according to an embodiment of the present invention, which incorporates a transparent elastomer layer (e.g., PDMS), looks almost identical to a sheet of hydrogel. However, the hydrogel laminates of the present invention possess a set of properties and capabilities not achievable in existing hydrogels: (a) the hydrogel laminates have high water content, slippery surfaces, and can be functionalized with biomolecules in the same fashion as common hydrogels, (b) the laminates are robust, highly stretchable, and impermeable to molecules of various types and sizes across the structure, similar to bulk elastomer sheets, (c) the modulus of the laminate (along in-plane directions) can be tuned by varying the types of elastomer, type and composition of the hydrogel layers, and the thicknesses of the different layers, (d) different sensing or stimuli-responsive molecules/mechanisms can be incorporated in different hydrogel layers of the laminate (e.g., as illustrated in FIG. 1B) to monitor environmental conditions on different sides of the laminate (e.g., pH, temperature, and biomolecule concentration), (e) a variety of different types of therapeutic agents and functional molecules can be incorporated into different hydrogel layers of the laminate and released independently from different sides of the laminate (e.g., as shown in FIG. 1C).

A general protocol for fabrication of the hydrogel laminates with an elastomer layer is as follows. It is noted that a similar protocol applies for hydrogel laminates with other substrate (i.e., plastics, glass, ceramics and metals). A pristine elastomer sheet is first activated, followed by coating of the activated sheet with a hydrogel precursor solution, and curing of the hydrogel precursor solution to form the hydrogel coating. This procedure may be carried out on individual sides of the elastomer sheet until a chosen number of sides of the elastomer sheet are coated with a desired number of hydrogel layers to form the laminate structure. Alternatively, if desired, multiple sides of the elastomer sheet (or the substrate) mat be coated in a single process. For example, a pristine elastomer sheet may be first activated by immersion in a suitable activating solution (e.g., a benzophenone solution in ethanol or the like), followed by rinsing (e.g., with isopropanol or the like), and drying (e.g., drying with compressed nitrogen or the like). A hydrogel precursor solution containing both stretchy and dissipative components (as described above), along with optional sensing/stimuli-responsive and/or releasable molecules, may then be disposed on the activated elastomer sheet. Typically, the precursor solution is disposed on one activated surface (generally the top surface) of the elastomer sheet (unless multiple sides of the elastomer/substrate are coated in the same process). The elastomer with the layer of hydrogel precursor solution disposed thereon is then covered with a glass plate and cured, preferably by placement in a UV oven. Upon exposure to UV light (preferred UV-A, 315-400 nm or UV-B, 280-315 nm), the adsorbed activating molecules (e.g., benzophenone molecules) on the elastomer sheet act as free radical sources and grafting agents to the elastomers. In some embodiments, heat may alternatively be used to cure the hydrogel and graft the polymer chains to the elastomer/substrate, particularly for glass, metal and ceramic substrates. The hydrogel precursor is thereby cured to form a first hydrogel layer on one surface of the elastomer sheet/substrate (or on multiple surfaces if multiple surfaces are coated in a single process).

After curing of the first hydrogel layer, if a second hydrogel layer is to be formed on the opposite side of the elastomer (and the second hydrogel layer was not formed at the same time as the first hydrogel layer), then the assembly (the elastomer/substrate with first hydrogel layer disposed thereon) may be suitably positioned (e.g., flipped over) to coat the opposite side with the second hydrogel layer. In particular, the exposed surface of the elastomer/substrate (i.e., surface not coated with the first hydrogel layer) is activated, followed by coating of the activated surface with a hydrogel precursor solution, and curing of the hydrogel precursor solution to form the second hydrogel coating. For example, as with the first hydrogel layer, the surface may be treated with benzophenone or the like to activate the second surface. Hydrogel precursor solution, containing both stretchy and dissipative components, along with optional sensing, stimuli-responsive and/or releasable molecules, is then placed on the treated surface and covered with a glass plate. The hydrogel is cured to form a second hydrogel layer on the elastomer/substrate. The result is a hydrogel-elastomer/substrate-hydrogel structure with robust hydrogel layers strongly bonded on opposing sides of the elastomer sheet/substrate. If desired, the thickness of the first and second hydrogel layers can be controlled, for example, by using spacers of different thicknesses.

The unique capabilities of the present invention hydrogel laminates to spatially control environmental sensing and drug release provides advantages in a variety of applications, particularly in the healthcare industry in which these properties play a major role. For example, the present hydrogel laminates can be used in wound and dermal care, as well as internal applications such as gastrointestinal and urinary tract treatments. For instance, hydrogel-coated catheters could be used to deliver anti-inflammatory drugs to the urethra while monitoring biomolecules present in the urine. The presence of hydrogel surfaces would also beneficially decrease friction, which would reduce irritation and discomfort of the catheters.

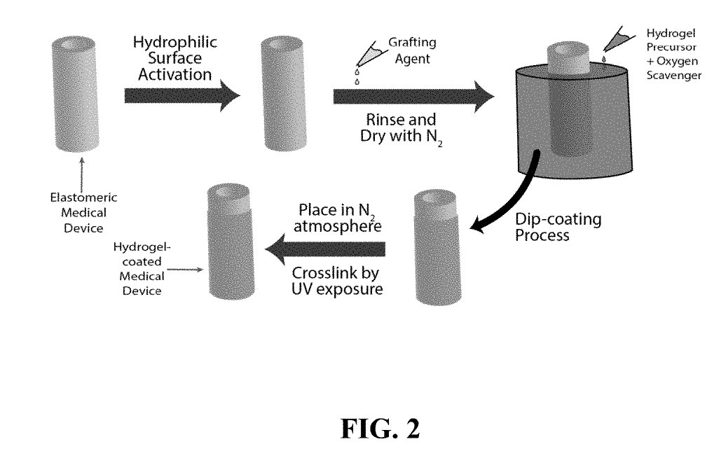

According to embodiments of the present invention, the outer surfaces of medical devices are coated using a modified hydrogel laminate coating technique. FIG. 2 depicts a general coating strategy for coating a medical device or other structure. Prior to coating with hydrogel precursor solution, the surface of the device is pre-treated. Pre-treatment includes steps to increase the hydrophilicity of the device surface (e.g., plasma treatment, corona discharge treatment, chemical modifications) and incorporation of grafting agent (e.g., heterobifunctional silanes, benzophenone, surface-bound ATRP initiators, or the like). The grafting agent will be adsorbed to the surface and enable grafting of the hydrogel polymer chains to the surface. The activated device is then rinsed and dried, followed by dip coating in a hydrogel precursor solution. The hydrogel precursor is a solution containing a polymerizable monomer (e.g., acrylamide, polyethylene glycol diacrylate, hydroxyethyl methacrylate or derivatives), an energy-dissipative component e.g., (polyvinyl alcohol, alginate, chitosan, hyaluronate or other high molecular weight polymers), the respective crosslinker molecule for each network, oxygen scavenger compounds (e.g., glucose oxidase, riboflavin, etc.), photoinitiator (e.g., Irgacures, phosphine oxides, aromatic ketones, etc.), releasable molecules (e.g., therapeutic agents, imaging agents, etc.) and/or sensing molecules or components. The viscosity of the precursor solution can be modified to achieve hydrogel coatings of different thicknesses. The hydrogel precursor coating is then crosslinked, preferably in an inert atmosphere (nitrogen or similar gas) under UV exposure. The hydrogel coated medical device can then be subjected to any desired post-treatment, such as removal of unreacted molecules and byproducts, achieving full swelling and incorporating sensing or releasable molecules.

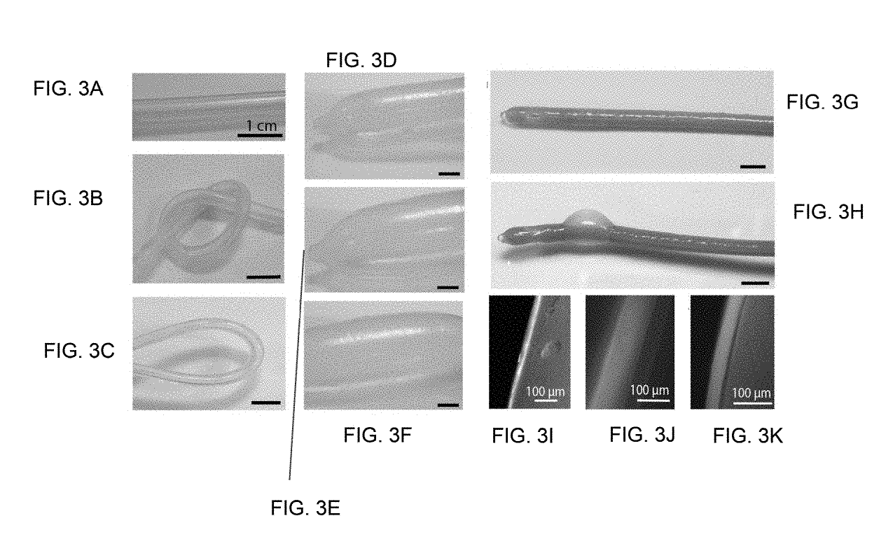

FIGS. 3A-K illustrate several medical devices that were coated with a hydrogel in accordance with the present invention. In particular, silicone tubing (VWR International, stained by immersion in green food dye solution—FIGS. 3A-C), a latex condom (Trojan®—FIGS. 3D-F), and a latex Foley catheter (Bard Medical—FIGS. 3G-H) were coated with a thin and uniform hydrogel layer.

As an example, the Foley catheter may be coated with chitosan-acrylamide through the use of a hydrogel precursor which is prepared by mixing chitosan (Sigma-Aldrich 740500), acrylamide, acetic acid, Irgacure I-2959, N, N-methylenebisacrylamide, sodium tripolyphosphate, glucose and glucose oxidase. The catheter is first cleaned (e.g., with isopropanol and plasma treatment for about 1 min/30 W at a pressure of 350 mtorr). The cleaned catheter is immersed in an activating solution (e.g., benzophenone solution) for a suitable time (e.g., about 2 min) to activate the surface of the catheter. Following activation, the catheter is rinsed and dried (e.g., rinsed with isopropanol and dried with nitrogen). The catheter is dip-coated in the hydrogel precursor and placed in a nitrogen environment. Crosslinking of the hydrogel may be done in a UV oven (e.g., for 60 min with 8 W power and 365 nm). Any unreacted molecules and byproducts may then be removed from the coated catheter (e.g., by immersion in sodium tripolyphosphate solution for an extended period, e.g., approximately 24 h).

The hydrogel coatings as formed are robust and strongly bonded to the tubing (FIGS. 3A-C). In particular, the hydrogel coatings of the present invention resist physical manipulation (stretching, bending, folding into a knot) without delamination and failure, as depicted in FIGS. 3B-C. Further, as shown in FIGS. 3E, 3F and 3H, the condom (FIGS. 3D-F) and catheter (FIG. 3G-H) having the present invention hydrogel coating thereon can be inflated to maximum size without delamination and failure of the hydrogel coating.

Further, by dip-coating the devices in the hydrogel precursor solution, the resulting hydrogel layer is made very thin, as shown in the cross-sectional images in FIGS. 3I-K. In FIGS. 3I-K, the hydrogel layer was stained by immersing in fluorescein solution, and it appears white (high fluorescence intensity) to the left of the elastomeric device (gray). The scale bars are all 1 cm unless indicated otherwise.

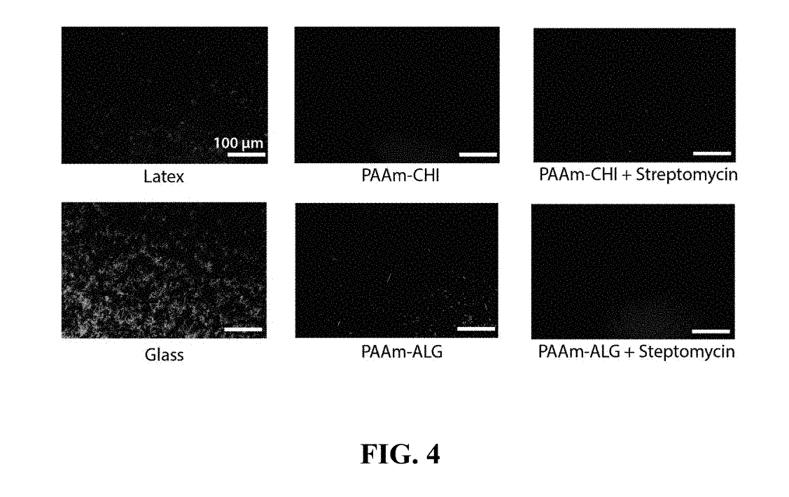

These results demonstrate that the present hydrogel coatings and techniques can be successfully utilized to create medical devices based on hydrogel-elastomer laminates. The high robustness and stretchability of the hydrogel-elastomer laminates allows for proper functioning of the devices under stresses and deformations. The tough hydrogel coatings are further biocompatible and can prevent bacterial adhesion, which is shown in FIG. 4, in which controls (latex and glass) exhibit large amounts of bacteria after a 24 hour incubation period while the hydrogel laminates show minimal bacterial adhesion. As further depicted in FIG. 4, the use of chitosan as a hydrogel layer, and the addition of antibiotic, improve the antibacterial action of the hydrogel laminates. In addition, the lubricious and highly hydrated hydrogel coating surfaces provided by the present invention are capable of improving protein adsorption, anticlotting and foreign-body reaction of the device as compared to an uncoated device. Moreover, the coating protocol of the present invention can be designed to include therapeutic agents in the hydrogel coating and/or to introduce hydrogel components with known biological responses such as PEG, hyaluronic acid, and zwitterionic groups.

The hydrogel coatings of the present invention can also be beneficially utilized as coatings for substrates that are utilized in underwater applications, such as boats and other equipment. In such applications, the substrate might be exposed to bacteria, algae, protozoans, and animals (zooplankton, tube worms, mussels, barnacles). By providing the present invention hydrogel coating on the substrate surface (such as steel and aluminum alloys for ship hulls), the surface friction and overall hydrodynamic drag on vessels and lines can be decreased. The highly hydrated and lubricious surface inhibits the attachment of fouling organisms (as compared to the uncoated substrate) and allows for easier removal of the fouling material. In addition, one or more functional molecules may be incorporated into the hydrogel coating layer for release into the surrounding water as needed, such as biocides to eliminate any fouling organism attached to the coating.

The hydrogel coatings and methods of the present invention will be further illustrated with reference to the following Examples which are intended to aid in the understanding of the present invention, but which are not to be construed as a limitation thereof.

### EXAMPLES

### Fabrication of Impermeable Hydrogels

All chemicals used in the following Examples were purchased from Sigma-Aldrich and used as received, unless indicated otherwise. The impermeable hydrogel laminates were made by bonding two hydrogel layers containing either acrylamide-alginate (AAm-ALG) or acrylamide-chitosan (AAm-CHI) to a thin elastomer sheet.

The acrylamide-alginate hydrogel was made by mixing 1 mL of a previously degassed aqueous pre-gel solution (12 wt % acrylamide (Sigma-Aldrich A8887), 2 wt % sodium alginate (Sigma-Aldrich A2033)) with 60 μL of 0.2 wt % N,N′-methylenebisacrylamide (BIS; Sigma-Aldrich 146072), 10 μL of 0.2 M ammonium persulfate (APS; Sigma-Aldrich A3678), 20 μL of 1.0 M calcium sulfate (Sigma-Alginate C3771), and 1.0 μL of N,N,N′,N′-tetramethylethylenediamine (TEMED; Sigma-Aldrich T9281).

The acrylamide-chitosan hydrogel was made by mixing 1 mL of a previously degassed aqueous pre-gel solution (18 wt % acrylamide, 2 wt % chitosan (Sigma-Aldrich 740500), 2 wt % acetic acid (Sigma-Aldrich 27225)) with 60 μL of 0.2 wt % BIS, 15 μL of 0.2 M APS, 60 μL of 0.5 M sodium tripolyphosphate (Sigma-Alginate 238503), and 1.0 μL of TEMED.

The elastomer sheets were cut from a latex roll (McMaster Carr), rinsed with isopropanol (Sigma-Aldrich W292907), and dried with a stream of nitrogen gas. Then, a 10 wt % benzophenone (Sigma-Aldrich B9300) solution in ethanol (Sigma-Aldrich 459844) was placed on the top surface for 1 min, and the sheet was rinsed with isopropanol and dried. The hydrogel pre-gel solution was quickly placed on top of the latex sheet and covered with a chlorosilane-treated (GlassFree, National Diagnostics) glass plate. Then, the assembly was placed in a UV oven (365 nm UV; UVP CL-1000) for 1 h to cure. The assembly was removed from the oven and flipped. The exposed latex surface was rinsed with isopropanol and treated with 10% benzophenone solution in ethanol, and dried with nitrogen. More hydrogel pre-gel precursor was poured, covered with a glass plate, and cured for another hour. The laminate was removed from the glass plates, and imaged and tested as prepared or after immersion in 1×PBS for 24 h.

### Hydrogel Coating of Medical Devices

Silicone medical tubing (VWR International) was cut into ≈10 cm segments, rinsed with isopropanol (Sigma-Aldrich W292907), and dried with a stream of nitrogen gas. Then, individual segments were oxygen-plasma-treated for 45 s (30 W at a pressure of 350 mTorr; Harrick Plasma PDC-001), submerged for 60 s in a 10 wt % benzophenone (Sigma-Aldrich B9300) solution in ethanol (Sigma-Aldrich 459844), rinsed with isopropanol, and dried. The tubing segments were then dip-coated in a modified acrylamide-chitosan hydrogel precursor solution (composition detailed below) and cured by UV exposure (365 nm UV; UVP CL-1000) for 1 h inside a custom-made with a glass cover filled with nitrogen. After curing, the coated tubing was rinsed in PBS for 1 h before imaging. The catheter (Bard Medical BARDIA Latex Foley Catheter, 16 Fr.) and condom (Trojan ENZ) were coated using the same protocol but leaving the devices intact.

The modified acrylamide-chitosan hydrogel was made by mixing 1 mL of a previously degassed aqueous pregel solution (18 wt % acrylamide, 2 wt % chitosan, and 2 wt % acetic acid) with 40 mg of glucose (Sigma-Aldrich G5767), 30 μL of 1.0 wt % glucose oxidase (Sigma-Aldrich G2133), 60 μL of 0.2 wt % BIS, 15 μL of 0.2 M APS, 60 μL of 0.5 M sodium tripolyphosphate, and 1.0 μL of TEMED.

### Imaging of Coated Medical Devices

The coated devices were cut with a sharp razor blade and immersed in a 1×10⁻³M fluorescein (fluorescein sodium salt, Sigma-Aldrich 46960) aqueous solution for 1 min. The images were obtained using the built-in camera of a Nikon Eclipse LV100ND fluorescent microscope.

### Mechanical Testing

All uniaxial tensile tests were conducted in ambient air at room temperature. The tests took place in a few minutes, so the change of properties due to dehydration was not significant. Pure hydrogel, elastomer, and impermeable hydrogel laminate samples were cut (approximate size: 5 cm×1 cm) and stretched using a universal mechanical testing machine (2 kN or 20 N load cells; Zwick/Roell Z2.5) by gripping directly to the fixtures. The grip-to-grip separation speed was set to 20 mm min⁻¹ for all tests, resulting in a nominal strain rate of 1.0 min⁻¹. All tests were done in triplicate.

### Coefficient of Friction Testing

All tests were conducted at room temperature using a controlled stress rheometer AR-G2 (TA Instruments, New Castles, Del., USA) in normal force control mode with 20 mm steel parallel plate fixtures. Hydrogel, elastomer, and impermeable hydrogel laminate (approximate size: 4 cm×4 cm) samples were cut and placed in between the fixtures for testing. After placing the solvent trap, filling with water to minimize dehydration and zeroing the forces, a normal force of 0.4 N was applied to the sample. The sample was allowed to equilibrate for 10 min, then a set shear rate (0.1, 0.5, 1.0 s⁻¹) was established. After a 10 min equilibration interval, torque was measured over a 10 min interval. Following published methodology, the friction force (F_{R}) was calculated using Equation (1), below, where T is the torque and R is the radius of the parallel plate fixture. The COF was obtained by dividing the time-averaged friction force over the time-averaged normal force (N), as shown in Equation (2). All tests were done in triplicate, and the standard deviation was reported

$$\begin{matrix}{F}_{R} = \frac{4 T}{3 R} & ( 1 ) \\ COF = \frac{\overline{{F}_{R}}}{\overline{N}} & ( 2 )\end{matrix}$$

### Diffusion, Release, and Sensing Testing

A two-chamber diffusion device was made using cast acrylic plates (McMaster Carr), and small magnetic stir bars and stir plates (VWR International) were used for stirring. For the diffusion tests, a 3cm×3 cm hydrogel, elastomer, or impermeable hydrogel laminate sample was placed between the chambers and screwed together tightly to prevent leakage from the chambers. A 5.0×10−4 M rhodamine B solution (Sigma-Aldrich R6626) was placed on one chamber while DI water was placed in the opposite chamber, and 1 mL aliquots were taken every 10 min and placed in a disposable cuvette. Absorbance was measured using a spectrophotometer (BioMate 3S, ThermoFischer Scientific) and converted to concentration using calibration curves of solutions with known concentrations. The setup for release experiments was identical except both chambers initially contained DI water with rhodamine B and green food dye (Fast green FCF, Sigma-Aldrich F7252) being released from the hydrogel. For rhodamine diffusion and release, a wavelength sweep was performed to determine the maximum absorbance, which occurred at 550 nm. The calibration curve was constructed by measuring absorbance of standard solutions in the 0-50×10−6 m range. For green food dye release, the maximum absorbance occurred at 630 nm. The calibration curve was constructed by measuring absorbance of standard solutions in the 0-200 ppm range.

To calculate the diffusion coefficient of the PAAm-ALG hydrogel, a simple pseudo-steady state was employed as the chamber volume was much larger than the hydrogel sample volume and the characteristic diffusion time scale across the hydrogel membrane (t_{D}=L²/D≈3 min) was smaller than the experiment time scale (≈120 min). Therefore, the transient contribution of the diffusion equation can be neglected. Moreover, due to the geometry of the system, the diffusion can be assumed to be unidimensional, so the full diffusion equation can be simplified to:

$$D \frac{{\partial }^{2} c}{\partial {x}^{2}} = \frac{δ c}{δ t} = 0 \qquad ( 3 )$$

The solution to this equation is the linear expression as shown below, where C_{L} and C₀ are the downstream and upstream concentrations, and L is the thickness of the sample. However, since c₀ is much higher than C_{L} throughout the experiment, the solution can be further simplified to:

$$c ( x ) = {c}_{0} + \frac{{c}_{L} - {c}_{0}}{L} x \approx {c}_{0} ( 1 - \frac{x}{L} ) \qquad ( 4 )$$

The flux of dye (J) was calculated from Fick's first law of diffusion (J=−D (dc/dx)) and equated to the flux corresponding to the change in concentration of the downstream chamber (J=c_{L}V_{D}/At), where V_{D} is the chamber volume, A is the sample area normal to the diffusion direction, and t is the time. This yields Equation (5)

$$\frac{{c}_{L} {V}_{D}}{A t} \approx KD \frac{{c}_{0}}{L} \qquad ( 5 )$$

In this expression, K is the dye partition coefficient. By taking the time derivative of this expression and rearranging, Equation (6) is obtained, which indicates that the slope of normalized concentration (c_{L}/c₀) versus time will be directly proportional to the diffusion coefficient:

$$\frac{d}{d t} ( \frac{{c}_{L}}{{c}_{0}} ) \approx \frac{KDA}{{LV}_{D}} \qquad ( 6 )$$

For the rhodamine B diffusion experiment, the measured value of KD=1.80×10⁻⁷ cm² s⁻¹. Assuming K≈1, the value was in good agreement with previously published diffusion coefficient values.

### Bacterial Adhesion Testing

An engineered Escherichi coli strain expressing green fluorescent protein was cultured in a 24-well plate above 8 mm round samples of latex, glass or swollen hydrogel laminate (PAAm-ALG and PAAm-CHI, as described previously). Streptomycin sulfate (Sigma-Aldrich S9137) was added to the two laminate formulations, at a final concentration of 0.1 wt %. After incubating at 37° C. for 24 h, the samples were removed, rinsed with PBS to remove free-floating bacteria, and imaged with a fluorescent microscope (Nikon Eclipse LV100ND).

### EXAMPLE 1

### MECHANICAL PROPERTIES

To characterize the in-plane mechanical properties of the present invention hydrogel-elastomer laminates and individual components, uniaxial tensile tests were carried out with a mechanical testing machine at a strain rate of 1.0 min ⁻¹. The chosen materials for the laminates were latex elastomer (McMaster Carr) and PAAm-ALG tough hydrogel as discussed above. The samples were analyzed as prepared or after soaking in 1X phosphate buffer saline (PBS; Sigma-Aldrich) for 24 hours.

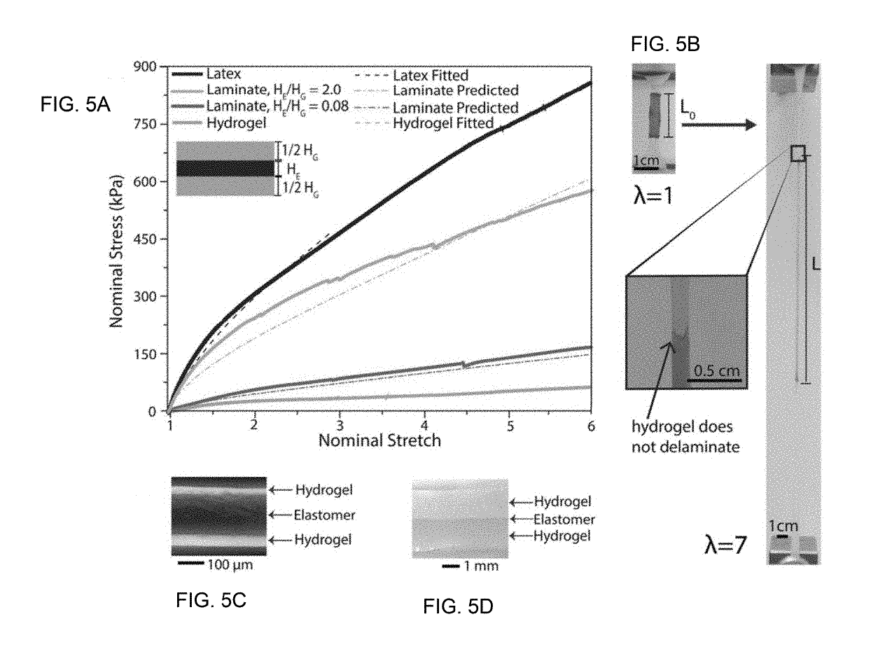

The solid dark blue and green curves in FIG. 5A show the nominal stress versus stretch curves of as-prepared single-material latex and PAAm-ALG hydrogel samples, where the nominal stress is the applied force over an undeformed cross-sectional area of the sample, and the stretch is the deformed length of the sample over its undeformed length. The latex underwent the same treatment as the hydrogel-elastomer laminate, but without addition of the hydrogel precursor. As demonstrated, both the latex and hydrogel samples were capable of high stretched without failure, but their tensile properties were dramatically different (as illustrated in FIG. 5B). In particular, the robust hydrogel-elastomer interface prevents delamination of impermeable hydrogel laminate (where the hydrogel is dyed green for visualization) upon stretching up to seven times. The shear modulus of latex was μ_{E}=166.0 kPa and that of hydrogel was μ_{H}=12.8 kPa, by fitting their stress-stretch curves to a neo-Hookean model. For undeformed laminates, the thicknesses of the elastomer layer and the combined hydrogels layers were denoted as H_{E} and H_{G}, respectively (FIG. 5A). Using the rule of mixture for composite materials, the shear modulus of the laminates was calculated via:

$$μ = \frac{{H}_{E}}{{H}_{E} + {H}_{G}} {μ}_{E} + \frac{{H}_{G}}{{H}_{E} + {H}_{G}} {μ}_{G} \qquad ( 7 )$$

Therefore, by varying the thickness ratio of elastomer and hydrogel layers in the laminate (i.e., H_{E}/H_{G}), the rigidity of the laminate were tuned significantly. When the elastomer/hydrogel thickness ratio was small (e.g., H_{E}/H_{G}=0.08, cross sectional image shown in FIG. 5D), the stress versus stretch curve resembled that of a hydrogel sample, as shown by the red solid curve of FIG. 5A. The broken red curve shows the predicted stress versus stretch curve from a neo-Hookean model using the shear modulus calculated using Equation (7). Conversely, if elastomer/hydrogel thickness ratio was large (e.g., H_{E}/H_{G}=2.0, cross-sectional image shown in FIG. 5C, wherein the hydrogel layers are stained with fluorescent dye for visualization), the stress versus stretch curve was similar to that of the elastomer, as shown by the light blue curve in FIG. 5A. Again, a neo-Hookean model was used to predict the broken light blue curve.

According to embodiments of the present invention, to expand the modulus range of the hydrogel laminates, it is possible to use stiffer elastomers such as PDMS or gutta-percha rubbers, or more compliant hydrogels (featuring lower density of crosslinks). Furthermore, the present invention laminates feature very robust bonding between the elastomer and hydrogel layers. As demonstrated in FIG. 5B, no delamination was observed when stretching a sample of the laminate up to seven times of its original length. In addition to having tunable tensile properties, the laminates are expected to have low coefficients of friction (COFs) due to the presence of slippery, high-water-content hydrogel on the surfaces of the laminate.

Tribology measurements (following the methodology introduced by D. P. Chang, J. E. Dolbow, S. Zauscher, Langmuir, 2007, 23, 250) were carried out using an AR-G2 rotational rheometer in normal force control mode with parallel plate geometry. The coefficient of friction (COF) was calculated from the measured torque and normal forces and are summarized in Table 1.

TABLE 1
Coefficient of friction (COF) for different surfaces as a function
of shear rate

| Surface Pair | | | |
|---|---|---|---|
| Condition | 0.1 [s⁻¹] | 0.5 [s⁻¹] | 1.0 [s⁻¹] |
| Steel on wet latex | 0.269 ± 0.049 | 0.363 ± 0.144 | 0.535 ± 0.089 |
| Steel on hydrogel | 0.078 ± 0.008 | 0.096 ± 0.014 | 0.109 ± 0.013 |
| laminate | | | |
| Steel on hydrogel | 0.082 ± 0.018 | 0.106 ± 0.016 | 0.169 ± 0.023 |
| Latex on wet latex | 0.216 ± 0.017 | 0.291 ± 0.081 | 0.545 ± 0.165 |
| Latex on hydrogel | 0.082 ± 0.016 | 0.114 ± 0.020 | 0.145 ± 0.024 |
| laminate | | | |
| Hydrogel laminate | 0.022 ± 0.004 | 0.024 ± 0.001 | 0.023 ± 0.002 |
| on hydrogel laminate | | | |

In particular, utilizing a rheometer steel fixture as the top plate, the COF of the samples of hydrogel, wet latex, and hydrogel laminate (with latex as the elastomer layer and PAAm-CHI as the hydrogel layer, HE/HG=0.2) against steel was measured at three different shear rates (0.1 [s⁻¹], 0.5[s ⁻¹], and 1.0 [s⁻¹]). While the COF of all samples increases with increasing shear rate, the COFs of the hydrogel and hydrogel laminate were identical (within experimental error) and consistently 3-5 times lower than the COF of wet latex. Latex was then attached to the rheometer fixture and the COF of latex against samples of hydrogel laminate, and wet latex was measured. The same trend was observed as the COFs were 2-4 times lower (across all tested shear rates) in the hydrogel laminate than in the wet latex. Furthermore, the measured COF between two hydrogel laminates was even lower than the COFs of steel on hydrogel laminates and latex on hydrogel laminates across different shear rates. This measured COF (between two hydrogel laminates) was in agreement with the reported COF values between swollen hydrogels. These results, which are expected to be independent of H_{E}/H_{G}, prove that the hydrogel laminates possess slippery surfaces similar to the corresponding hydrogels.

### EXAMPLE 2

### DIFFUSION PROPERTIES

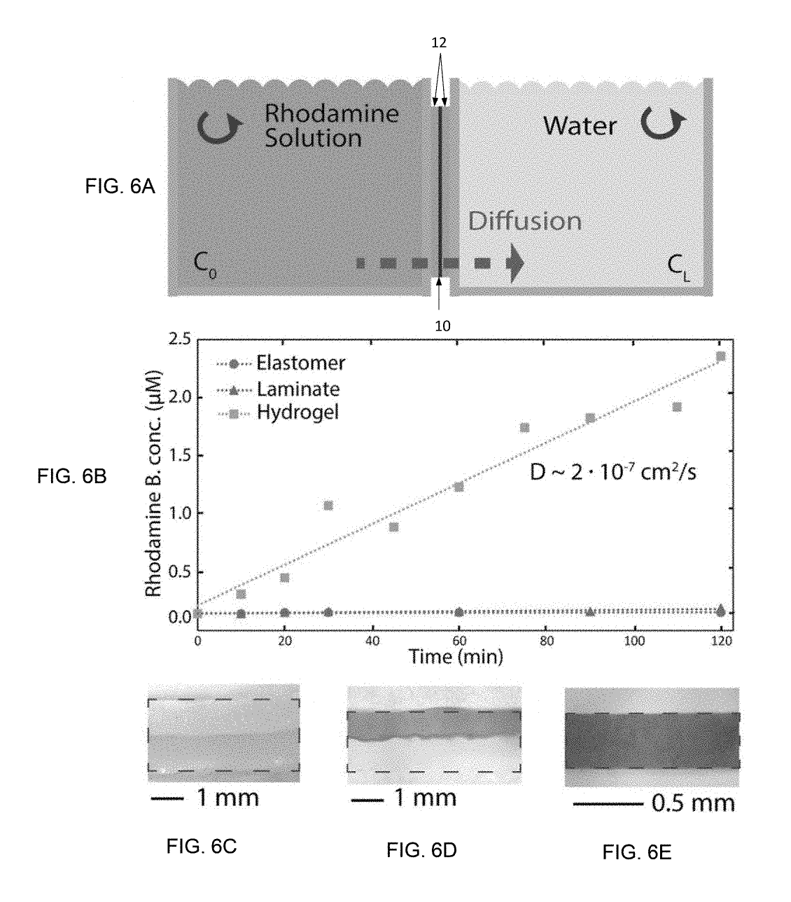

The impermeability of hydrogel-elastomer laminates was tested through a set of diffusion, release and stimuli-response tests. To examine the diffusion properties of the hydrogel laminates, a two-chamber diffusion device, shown in FIG. 6A, was employed. Samples of hydrogel (PAAm-ALG), elastomer (latex), or the corresponding hydrogel laminate (PAAm-ALG and latex, HE/HG=0.05) were placed between the two chambers containing a 5.0×10⁻⁴ m Rhodamine B (Sigma-Aldrich) solution and deionized water (DI water, Millipore). The concentration of rhodamine B diffusing to the water chamber was monitored by measuring the absorbance at 550 nm on a spectrophotometer and converting this result to concentration using a calibration curve for known rhodamine B concentrations. For the hydrogel sample, rhodamine B readily diffused to the water chamber (FIG. 6B, green data). This process can be described by a pseudosteady state, 1D diffusion model, which predicts a linear relationship between time and the concentration of rhodamine B in the water chamber.

The diffusion coefficient, calculated from the slope of the fit line (dashed green curve), was 1.80×10⁻⁷ cm²s⁻¹ and is in agreement with previously-reported values. For both elastomer and hydrogel laminate samples, there was no measurable diffusion of rhodamine to the water chamber over a 2 hour period (FIG. 6B, blue and red data), which demonstrated the impermeability of the laminate. Cross-sectional images of the hydrogel laminate and individual hydrogel samples after the diffusion test (FIGS. 6D and 6E, respectively) provide a visual confirmation of this result. The diffusion of dye occurred readily across the hydrogel, but was halted at the elastomer layer in the middle of the laminate.

### EXAMPLE 3

### RELEASE PROPERTIES

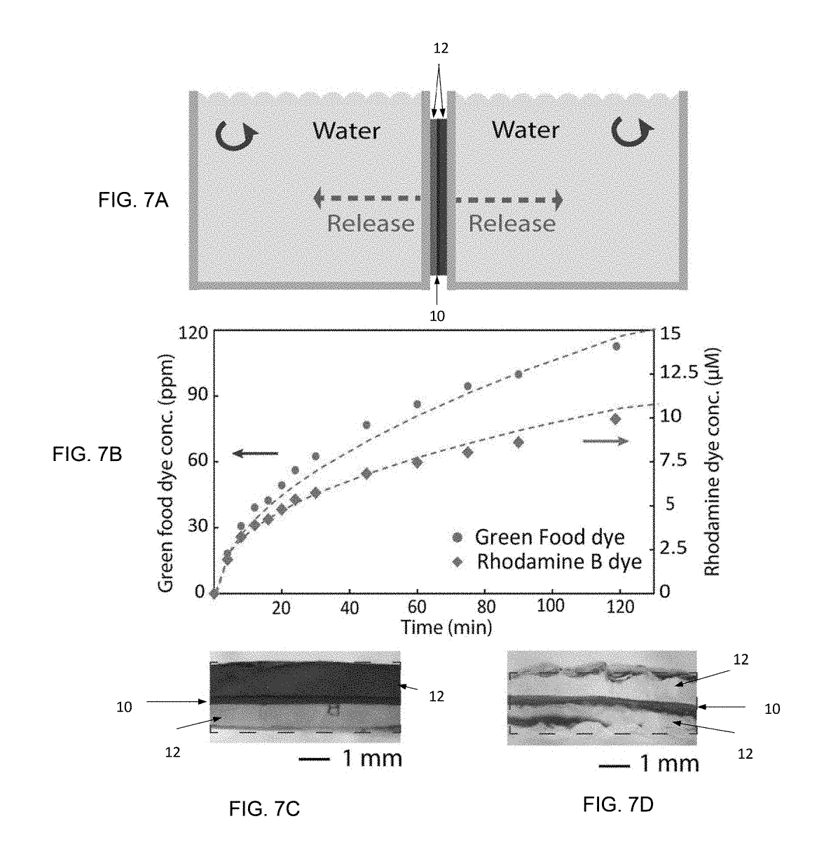

Taking advantage of the impermeability of the hydrogel-elastomer laminate, the possibility of releasing different molecules from the two sides of the hydrogel sheets in the laminate was tested. Green food dye (Fast Green FCF, Sigma-Aldrich) and rhodamine B were used as model drugs and were loaded into the opposing two hydrogel sheets in the hydrogel-elastomer laminate structure. As shown in FIG. 7A, this laminate was placed in the diffusion apparatus initially containing DI water in both chambers. The concentrations of green food dye and rhodamine B in the solutions of both chambers were measured over time using absorbance measurements. The results demonstrated typical power law release profiles for both drugs over the course of the experiment (FIG. 7B), indicating Fickian release from the hydrogel layers in the laminate (exponent 0.5). The cross-sectional pictures of the laminate before and after the experiment (FIGS. 7C and 7D, respectively) provided a visual demonstration of the dye release. Since the dyes used have absorbance maximums at different wavelengths (550 nm and 630 nm), it was possible to detect dye leakage through the impermeable hydrogel-elastomer laminate by monitoring the absorbance signals at the two wavelengths in both chambers. No green food dye was detected in the rhodamine chamber and vice versa, further validating the impermeability of the hydrogel-elastomer laminates, and the possibility of spatially controlled release from two sides of the hydrogel-elastomer laminate.

### EXAMPLE 4

### SENSING PROPERTIES

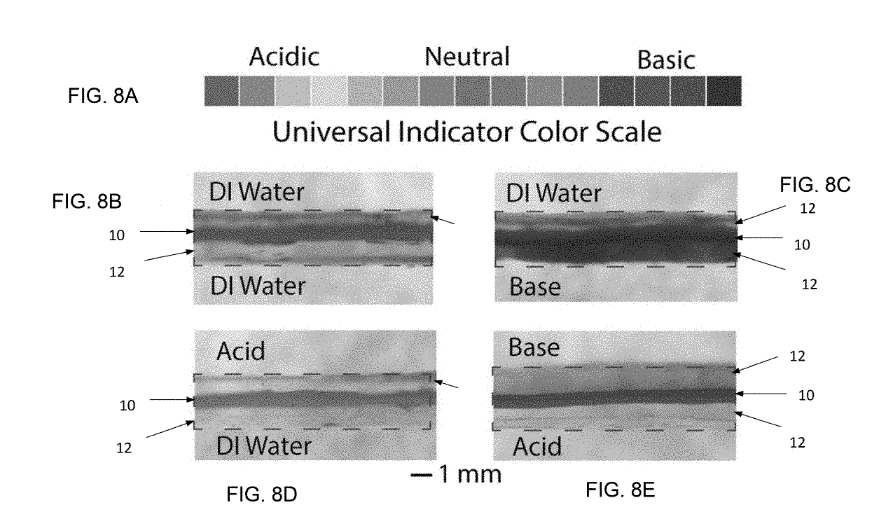

In addition to releasing molecules of interest to separate environments, the impermeable hydrogel laminates of the present invention also enable sensing of different types of stimulus, or different conditions of the same stimulus on the two sides. To demonstrate this capability, a universal indicator solution (Sigma-Aldrich) was added to the hydrogel layers as a visual pH indicator. Using the diffusion apparatus, the laminates were contacted for 3 min with solutions of different pH on the two sides and cross-sectional images were obtained. The resulting cross-sectional images can be seen in FIG. 8B-E. As demonstrated, there were clear visual differences in the hydrogel layers depending on the pH of the contacting solutions. The nonpermeable nature of the overall hydrogel-elastomer laminate structures combined with the fast diffusion of small molecules within the hydrogel layers allowed for fast sensing of different pH environments on the two sides of the nonpermeable hydrogel-elastomer laminate sheet. This capability can thus be utilized to sense biologically relevant parameters, such as glucose, pH, ion concentration, or protein levels in separate environments.

The present invention hydrogel coatings and methods provide numerous advantages. The tough hydrogel coating material provides a device that can be handled, inserted and manipulated without rupture of the coating material. The formation of robust interfaces between the device and coating provides interfacial toughness values above 1000 J/m² in contrast with current coatings and methods which provide less robust interfaces susceptible to coating delamination and device failure upon stress and swelling of the coating. The present hydrogel coatings further have controllable thickness, which makes it possible to tune the release kinetics of the releasable therapeutic agents while maintaining an ultra-low friction surface. The combination of features and customizable characteristics of the present hydrogel coatings and methods allows for the fabrication of tunable, functional hydrogel-based medical devices that are further infection-resistant and lubricious. The hydrogel coatings can be used for therapeutic agent release and/or sensing applications, while introducing user comfort due to the ultralow surface friction provided. As such, the present invention provides numerous benefits in the healthcare industry, particularly enabling robust hydrogel coating of a wide variety of medical devices (e.g., catheters, condoms, medical tubing, endoscopes, rubber examination gloves, etc.).

What is claimed is:

1. A hydrogel-substrate laminate comprising:
a layer of substrate having a top surface and a bottom surface, the substrate selected from elastomers, plastics, glass, ceramics and metals;
at least one hydrogel coating layer disposed on one or more of the top surface and the bottom surface of the substrate; and
one or more therapeutic agent disposed within the at least one hydrogel coating layer, and/or one or more sensing material disposed within the at least one hydrogel coating layer.

2. The hydrogel-substrate laminate according to claim 1, wherein a first hydrogel coating layer is disposed on the top surface of the substrate layer, and a second hydrogel coating layer is disposed on the bottom surface of the substrate layer.

3. The hydrogel-substrate laminate according to claim 2, wherein the layer of substrate is impermeable to small molecules, a first therapeutic agent is disposed within the first hydrogel coating layer, and a second therapeutic agent different than or the same as the first therapeutic agent is disposed within the second hydrogel coating layer.

4. The hydrogel-substrate laminate according to claim 2, wherein the layer of substrate is impermeable to small molecules, a first sensing material is disposed within the first hydrogel coating layer, and a second sensing material different than or the same as the first sensing material is disposed within the second hydrogel coating layer.

5. The hydrogel-substrate laminate according to claim 2, wherein the layer of substrate is impermeable to small molecules, at least one therapeutic agent is disposed within the first hydrogel coating layer, and at least one sensing material is disposed within the second hydrogel coating layer.

6. The hydrogel-substrate laminate according to claim 1, wherein at least on therapeutic agent and at least one sensing material are disposed within the at least one hydrogel coating layer,
wherein the at least one therapeutic agent and the at least one sensing material are in communication, and
wherein release of the at least one therapeutic agent from the at least one hydrogel coating layer is controlled by one or more environmental conditions sensed by the at least one sensing material.

7. The hydrogel-substrate laminate according to claim 6, wherein the at least one therapeutic agent is disposed within a first hydrogel coating layer, and the at least one sensing material is disposed within a second hydrogel coating layer, wherein the first and second hydrogel coating layers are separated from each other by the layer of substrate.

8. The hydrogel-substrate laminate according to claim 1, wherein the thickness of the hydrogel coating layer is predetermined to provide a hydrogel-substrate laminate having mechanical properties ranging from mechanical properties of the pure substrate to mechanical properties of the pure hydrogel.

9. The hydrogel-substrate laminate according to claim 1, wherein the at least one hydrogel coating layer comprises:
a physically crosslinked dissipative polymer network selected from PVA, collagen, gelatin, agar, agarose, dextran, alginate, hyaluronan and chitosan; and
a covalently crosslinked stretchy polymer network selected from polyacrylamide (PAAm), polyethylene glycol (PEG), polyethylene glycol derivatives, polyvinyl alcohol, poly-N,N-dimethylacrylamide (DMMA), polyacrylamide derivatives, and polyzwitterionic monomers.

10. The hydrogel-substrate laminate according to claim 8, wherein the substrate is an elastomer material, and the stretchy polymer network in the hydrogel coating layer is covalently grafted to elastomer chains of the elastomer material.

11. A hydrogel coated medical device comprising:
a medical device having an outer surface;
at least one hydrogel coating layer disposed on at least a portion of the outer surface of the medical device; and
one or more therapeutic agent disposed within the at least one hydrogel coating layer to provide therapeutic agent release into an environment surrounding the at least one hydrogel coating layer, and/or one or more sensing material disposed within the at least one hydrogel coating layer to provide sensing of one or more environmental conditions surrounding the at least one hydrogel coating layer.

12. The hydrogel coated medical device of claim 10, wherein the hydrogel coated medical device has a coefficient of friction less than the medical device without the hydrogel coating layer.

13. The hydrogel coated medical device of claim 10, wherein at least on therapeutic agent and at least one sensing material are disposed within the at least one hydrogel coating layer,
wherein the at least one therapeutic agent and the at least one sensing material are in communication, and
wherein release of the at least one therapeutic agent from the at least one hydrogel coating layer is controlled by one or more environmental conditions sensed by the at least one sensing material.

14. The hydrogel coated medical device of claim 10, wherein a plurality of different therapeutic agents are disposed within the at least one hydrogel coating layer so as to be independently released from the at least one hydrogel coating layer.

15. A method for fabricating a hydrogel-substrate laminate comprising:
activating at least one surface of a pristine substrate material, the substrate material selected from elastomers, plastics, glass, ceramics and metals;
coating at least one surface of the activated substrate material with a layer of hydrogel precursor solution;
curing the hydrogel precursor solution to thereby form a tough, double network hydrogel coating layer of controllable thickness firmly grafted on the at least one surface of the activated substrate material.

16. The method of claim 15, wherein the hydrogel precursor solution contains one or more therapeutic agent and/or one or more sensing material.

17. The method of claim 15, further comprising, prior to curing the hydrogel precursor solution, predetermining desired mechanical properties of the hydrogel-substrate laminate ranging from mechanical properties of the pure substrate to mechanical properties of the pure hydrogel, and based on the desired mechanical properties, determining a corresponding thickness of the hydrogel coating layer, and placing of at least one spacer on the layer of hydrogel precursor solution to control a thickness of the hydrogel coating layer.

18. The method of claim 15, wherein activating at least one surface of a pristine substrate material comprises surface functionalization of the substrate surface.

19. The method of claim 15, wherein the hydrogel coating layer is formed by grafting hydrogel polymer chains onto the substrate surface by generation of free radicals at the substrate-hydrogel interface.
